(19) United States
(12) Patent Application Publication
(10) Pub. No.: US 2006/0116557 A1
(43) Pub. Date: Jun. 1, 2006

(54) Methods and systems for evaluating patient data

(75) Inventors: Timothy J. Moore, Reno, NV (US); Ronald D. Geraty, Marblehead, MA (US); Aria DiBiase, Menlo Park, CA (US); John Tillotson, Santa Rosa, CA (US); Lester John Lloyd, Orinda, CA (US); Lauren F. Yazolino, Orinda, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: Alere Medical Incorporated

(21) Appl. No.: 11/001,667

(22) Filed: Nov. 30, 2004

(51) Int. Cl. A61B 5/00

(52) U.S. Cl. 600/300; 128/920

(57) ABSTRACT

Methods and systems for prioritizing patient data from a plurality of patients for review by a healthcare professional are provided. Embodiments of the subject methods include determining a priority score for each patient based on patient data data, and presenting the data to a healthcare professional for review in an order based on the determined priority scores. Systems for use in performing the subject methods are also provided.

### BACKGROUND OF THE INVENTION

Frequent monitoring of patients permits a healthcare professional such as a nurse and/or physician to detect worsening symptoms as they begin to occur, rather than waiting until a critical condition has been reached. As such, home monitoring of patients with chronic conditions is becoming increasingly popular in the health care industry for the array of benefits it has the potential to provide. Potential benefits of home monitoring are numerous and include: better tracking and management of chronic disease conditions, earlier detection of changes in the patient condition, and reduction of overall health care expenses associated with long term disease management. The home monitoring of a number of diverse “chronic diseases” is of interest, where such diseases include diabetes, dietary disorders such as anorexia and obesity, respiratory diseases, AIDS and other chronic viral conditions, conditions associated with the long term use of immunosuppressants, e.g. in transplant patients, asthma, chronic hypertension, chronic use of anticoagulants, and the like.

Home monitoring is being used to monitor patients with heart failure (HF), also known as congestive heart failure (CHF). HF is a syndrome in which the heart is unable to efficiently pump blood to the vital organs. Most instances of HF occur because of a decreased myocardial capacity to contract (systolic dysfunction). However, HF can also result when an increased pressure-stroke-volume load is imposed on the heart, such as when the heart is unable to expand sufficiently during diastole to accommodate the ventricular volume, causing an increased pressure load (diasystolic dysfunction). In either case, HF is characterized by diminished cardiac output and/or damming back of blood in the venous system. In HF, there is a shift in the cardiac function curve and an increase in blood volume caused in part by fluid retention by the kidneys. Indeed, many of the significant morphologic changes encountered in HF are distant from the heart and are produced by the hypoxic and congestive effects of the failing circulation upon other organs and tissues. One of the major symptoms of HF is edema, which has been defined as the excessive accumulation of interstitial fluid, either localized or generalized.

In the conventional management of HF patents, where help is sought only in crisis, a cycle occurs where patients fail to recognize early symptoms and do not seek timely help from their care-givers, leading to emergency department admissions (Miller, P. Z., 1995, “Home monitoring for congestive heart failure patients,” Caring Magazine, August 1995: 53-54). A prospective, randomized trial of 282 patients was conducted to assess the effect of the intervention on the rate of admission, quality of life, and cost of medical care. In this study, a nurse-directed, multi disciplinary intervention (which consisted of comprehensive education of the patient and family, diet, social-service consultation and planning, review of medications, and intensive assessment of patient condition and follow-up) resulted in fewer readmissions than the conventional treatment group and a concomitant overall decrease in the cost of care (M. W. Rich et al., New Engl. J. Med. (1995) 333:1190-95). Similarly, comprehensive discharge planning and a home follow-up program was shown to decrease the number of readmissions and total hospital charges in an elderly population (M. Naylor et al., Amer. College Physicians (1994) 120:999-1006). A recent trial of a daily electronic home monitoring system, the Weight Monitoring in Heart Failure (WHARF) Study, successfully demonstrated significant benefits of a HF home monitoring program on mortality (L. R. Goldberg et al., Am. Heart J. (2003) 146:705-712). The study was a multicenter, randomized trial of a technology-based daily weight and symptom monitoring system and enrolled 280 HF patients from 16 clinical sites. The study demonstrated a significant 56% reduction in the 6-month all-cause mortality in those HF patients randomized to the home monitoring system. Therefore, home monitoring is of particular interest in the HF management segment of the health care industry.

Home monitoring systems may include the remote monitoring of patients by a healthcare professional at a remote site and may involve the review of health-related information about a plurality of patients at a remote site, where the information may be reviewed in any order, e.g., a random order or first-in, first-out order. However, review of information about one or more monitored patients may indicate an emergent condition that may require immediate medical intervention. Due to the volume of patient data and review order of the data, patients presenting with emergent conditions may not necessarily be the first to be reviewed and may be reviewed after non-emergent patients. Depending on the number of patients reviewed and the time required to review data pertaining to each patient, a substantial amount of time may elapse before a healthcare professional reviews information about an emergent patient. This period of time may result in a significant delay in medical intervention and a worsening medical condition. In certain instances, this delay may be life threatening.

Accordingly, there continues to be an interest in the development of new monitoring systems. Of particular interest would be the development of systems that prioritize patient data from a plurality of patients for review by a healthcare professional.

### SUMMARY OF THE INVENTION

Methods and systems for prioritizing patient data from a plurality of patients for evaluation by a healthcare professional are provided. Embodiments of the subject methods include receiving data at a remote site from a plurality of patients, determining a patient status score for each patient based on the transmitted data, and presenting the data to a healthcare professional for evaluation in an order based on the determined patient status scores.

Embodiments also include methods of monitoring patients suffering from a condition (e.g., congestive heart failure) that include receiving data at a remote monitoring site from a plurality of patients suffering from a condition such as congestive heart failure, assigning a priority score to the data received from each patient and applying an algorithm to the data using the priority score to obtain a patient status score, and presenting the patient status score to a healthcare professional for evaluation.

Embodiments of the subject systems include a remote monitoring system that includes a remote site monitoring system that includes a processor configured to receive and process patient data to provide a patient status score for each patient based on the data, a plurality of patient data input modules in communication with the processor; and communication means configured to transmit data from each of the patient data input modules to the processor.

Embodiments also include computer programming for performing the subject methods, wherein the computer programming is embodied on a computer-readable medium.

### BRIEF DESCRIPTIONS OF THE DRAWINGS

FIG. 1 shows a flow chart of an exemplary embodiment of the subject methods.

FIG. 2 shows an example of applying an exemplary embodiment of a subject algorithm to data from two patients.

FIG. 3 shows a chart of exemplary priority scores.

FIG. 4 shows a schematic of an exemplary embodiment of a system according to the subject methods.

FIG. 5 shows an exemplary embodiment of an algorithm that maybe applied to a patient's weight.

### DETAILED DESCRIPTION OF THE INVENTION

As summarized above, the subject invention provides methods and systems for prioritizing patient data from a plurality of patients for evaluation by a healthcare professional. The subject invention may be used for monitoring a plurality of patients in which data about each patient is transmitted from a first site, such as a patient's residence or the like, to a second, remote site for evaluation, where the patient data may be presented for evaluation in a prioritized order based on the state of the patients' health as indicated by the data. In certain embodiments, computing means may perform the subject methods.

The subject invention is suited for remotely monitoring patients having a condition such as a medical condition, e.g., a medical condition that requires frequent or constant monitoring by a healthcare professional. The subject invention may be employed in the monitoring and management of patients having a variety of conditions, including medical conditions such as, but not limited to, immunosuppression (e.g., due to the chronic use of immunosuppressants as found in transplant patients), and disease conditions, such as but not limited to diabetes, nutrition disorders, e.g., anorexia and obesity, AIDS and other bacterial or viral associated conditions, asthma, chronic hypertension, chronic use of anticoagulants, and cardiac associated diseases, such as congestive heart failure (CHF).

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All patents and publications mentioned herein are incorporated herein by reference to disclose and describe the methods and/or materials in connection with which the patents and publications are cited. The citation of any patent or publication is for its disclosure prior to the filing date and should not be construed as an admission that the present invention is not entitled to antedate such patent or publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

The figures shown herein are not necessarily drawn to scale, with some components and features being exaggerated for clarity.

Methods

As described above, embodiments of the subject invention involve prioritizing a plurality of patients, i.e., patient records, so that they may be evaluated by a healthcare professional in a particular order, where the order is based at least in part on the health of the patients. In other words, the patients' data are prioritized into a specific order so that a healthcare professional may evaluate the patients in that order. In this manner, patients warranting immediate evaluation by a healthcare professional such as a nurse, e.g., emergent or urgent cases or other cases requiring immediate attention, may be presented to the healthcare professional for evaluation prior to patients who do not warrant immediate attention. Stated otherwise, patients who present with data that indicate health conditions that are worse relative to other patients are prioritized in a manner so that they may be evaluated by a healthcare professional prior to the other patients.

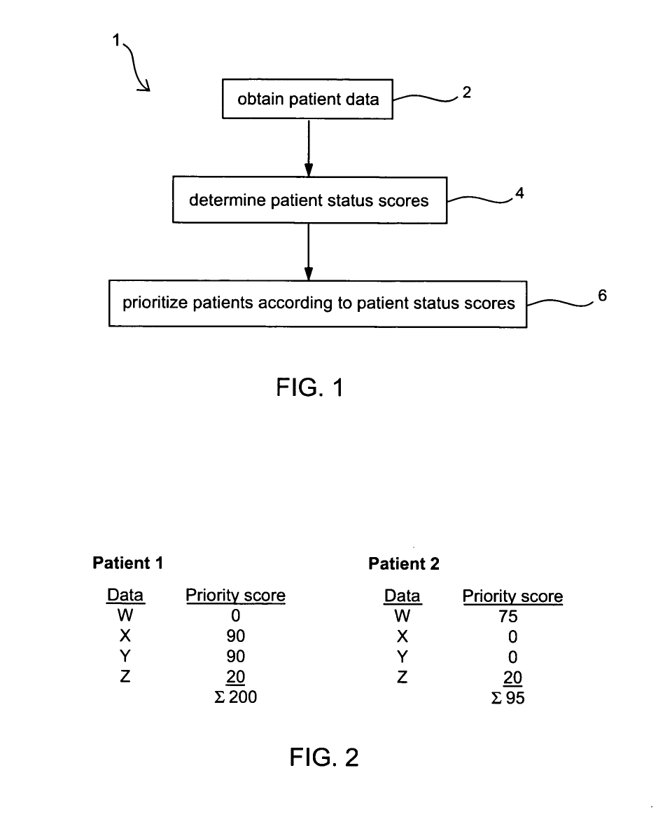

FIG. 1 shows a flow chart of an exemplary embodiment of a method 1 of the subject invention. Method 1 includes obtaining patient data 2 about a plurality of patients at a remote monitoring site, determining a patient status score for each patient 4, e.g., by applying an algorithm to the data, and prioritizing the patients according to the patient status scores 6. Once prioritized, the patients' data may be evaluated by a healthcare professional in the order based on the patient status scores. A “remote site” refers to a site having a location other than the location at which the patient is present, which may be a different room, building, city, state, country, etc. In further describing the invention, a healthcare professional is described primarily with respect a healthcare professional that is a nurse for convenience, where such description is not intended to limit the scope of the invention.

Data for each patient generally includes, but is not limited to, one or a plurality of diagnostic data points or parameters diagnostic of the health of the patients, i.e., one or more “diagnostic parameters”. A data point or parameter is diagnostic if it provides information about a patient's health. For example, data may include information about symptoms, medications, food intake, exercise routine, and the like. For example, the degree of edema, weight, blood pressure, heart rate, temperature, may be “diagnostic parameters” for certain indications. Data that correlates closely with a patient's condition, or any change in the patient's health, is usually chosen. For example, data for CHF patients may include their degree of edema. Sensitive measurement of weight changes correlates with edema, and may be used as a surrogate. Data for obese patients may include weight measurements, blood pressure, etc.

For certain indications, e.g., CHF, a plurality of different types of diagnostic parameters may be used to determine a patient status score for a patient. For example, in the case of CHF patients, diagnostic parameters may include, but are not limited to, measurement parameter data, and symptom parameter data, patient type parameter data, event parameter data, medication parameter data, co-morbidity or medical condition parameter data, and the like. For example, diagnostic measurement parameter data for CHF patients may include, but is not limited to, weight data (and/or edema information), symptom data, etc. Other data points may be included as well and will be known to those of skill in the art.

The data may be obtained in any suitable manner. In certain embodiments, data is transmitted to a remote monitoring site from a plurality of patients such that each patient being monitored transmits diagnostic data to a remote monitoring site. Data may be transmitted from each patient to a remote monitoring site using any suitable method.

For example, each patient may transmit data related to the patient's health, e.g., weight and symptoms, to a remote monitoring site, where the data may be received at the monitoring site for evaluation by a nurse. The data may be transmitted or received over any suitable period of time. For example, patient data may be transmitted from a patient to a remote monitoring site once per month or more or less frequently, e.g., bimonthly, once per week, bi-weekly, one or more times during about a 24 hour period, etc. In certain embodiment, a monitoring protocol may include receiving data transmitted from a patient at least about one time per day. For example, a remote monitoring site may receive data transmitted from monitored CHF patients about one time per day, e.g., in the morning. The data for CHF patients may vary, where data may include information related to a patient's health status such as weight information (and/or edema information) and/or other information about symptoms the patient may have. Such other information may include, but is not limited to, answers to health-related questions such as: (1) “Did you wake short of breath?”; (2) “Did you use an extra pillow?”; (3) “Are your feet more swollen?”; (4) “Are you coughing more than usual?”; (5) “Are you more breathless than normal?”; (6) “Did you exercise today?”; and the like. The questions may vary depending on the time of day in which they are asked, as certain questions may be more relevant in the morning than in the evening, etc.

Methods and systems for transmitting data such as weight and other information from a patient, such as a CHF patient and the like, to a remote monitoring site and which may be adapted for use in the subject invention are described, e.g., in U.S. Pat. No. 6,080,106, the disclosure of which is herein incorporated by reference.

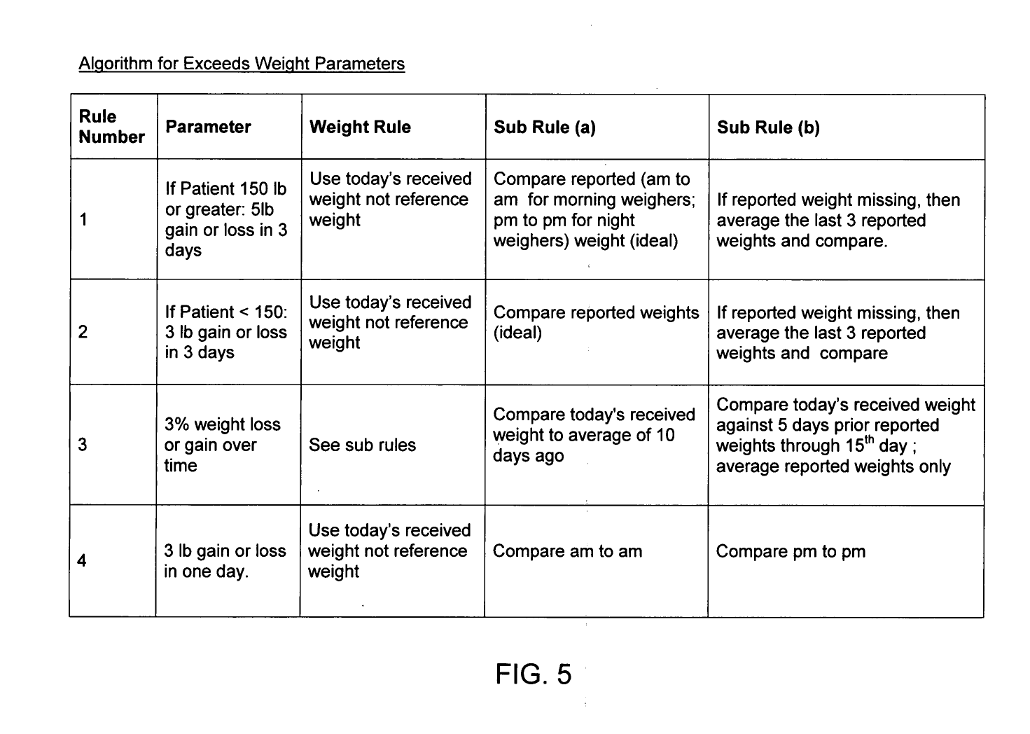

Regardless of the particular data transmitted from each patient, the data may be transmitted in any suitable form including raw and processed form. For example, one or more weight measurements may be transmitted, or a processed weight measurement may be transmitted, e.g., using a suitably programmed processor. A patient's weight may be communicated to a processor, e.g., at the patient's location, and processed according to an algorithm that determines the change in weight from the previous weight and/or whether the weight is above or below a predetermined amount, reference, or the like. For example, a determination of whether a patient's weight exceeds a predetermined weight may be determined using an algorithm that processes the patient's weight transmitted by the patient to provide a percent or net change in weight. This processed weight data may then be transmitted to the remote monitoring site. Alternatively, such data processing may be performed at the monitoring site, e.g., by a processor at the monitoring site. For example, an exemplary algorithm for processing a patient's weight is shown in FIG. 5.

In certain embodiments, data is collected from a patient and transmitted from the patient site to a remote computer monitoring system at the monitoring site, e.g., to a processor or database of the computing system such as a database of patient records. Data may be transmitted from a patient using any suitable protocol, e.g., computer readable storage medium, a modem, serial interface, LAN, WAN, wireless transmitter, MICROWIRE, I²C, +5 V serial, 1-Wire® interface, General Purpose Interface Bus (“GPIB”), RS-232, Peripheral Component Interconnect (“PCI”), memory mapped I/O and Universal Serial Bus (“USB”), etc.

In addition to, or instead of, data transmitted by each patient, e.g., data that may be transmitted each day from each patient to a remote site, additional data may be employed in the determination of a patient's status score. Such data may include data that does not necessarily require frequent patient input, i.e., not highly variable, e.g., from day to day or the like, and may include diagnostic data and/or other data determined to be relevant to a patient's health and/or monitoring thereof. As such, this type of data may be stored at the remote monitoring site, e.g., in a database of patient records of a computer system at the remote site or at least accessible from the remote monitoring site. Accordingly, a patient status score may include both data received at the remote monitoring site by transmission from each patient to the site and data present at the remote site, e.g., in the patients' record at the site, or known to a healthcare professional or staff thereof at the site.

Regardless of the particulars of the data, once patient data is obtained at a remote monitoring site, e.g., transferred to computer system thereof, a patient status score is determined for each patient 4. A “score” is used broadly to refer to a quantitative or qualitative value, e.g., a rating or valuation which may be expressed as a number, letter, word or phrase, etc. Each patient status score is generally based at least in part on the relative health status of the respective patient, which may be determined at least in part from the data obtained at the monitoring site. In determining the patient status scores, an algorithm is applied to each patient's data to provide a patient status score for each patient. In certain embodiments, a computer processor may be employed to determine the patient status scores, which processor includes computer programming embodying the algorithm. The processing of patient data using a computer processor is described primarily with respect to a processor for convenience, however it will be apparent that a plurality of processors may be employed.

Patient status scores may be determined using any suitable algorithm. Certain embodiments include weighting each patient's data, and summing the weighted data to provide a patient status score. Data from each patient may be scored by assigning numerical values (e.g., integers between about 1 and about 500, e.g., between about 1 and about 100) herein referred to as priority scores, to each patient's data, i.e., to each data point that makes-up the data for each patient. The priority scores may be weighted according to their impact on the patient's health and/or other factors such as the amount of time elapsed from the previous evaluation, etc., which may be relevant to the monitoring of the patient. As described above, patient data may include a plurality of parameters, e.g, weight data, symptom data, etc. As such, a priority score may be assigned to each data point that makes-up a given patient's data, which priority scores may then be summed or otherwise computed to provide a total score or patient status score for a patient.

FIG. 2 provides an exemplary embodiment of such scoring in which each data parameter is assigned a weighted priority score and the priority scores are summed to provide a patient status score. This particular embodiment is described with respect to two monitored patients and data for each of the two patients includes four parameters, where this description is for exemplary purposes only and is no way intended to limit the scope of the invention. It will be apparent that more patients and/or more or less data may be employed. The data may include information transmitted from each patient and/or information present at the remote monitoring site, i.e., present in a database that includes patients' records. Data obtained for both Patients 1 and 2 includes data parameters W, X, Y and Z, and each parameter is assigned a weighted priority score. For example, the data points may include a hospitalization parameter W which indicates whether the patient has been hospitalized within a certain period of time, e.g., within the past 30 days, and to which a priority score is assigned (in this particular example a priority score of 75) if the patient has been hospitalized within the predetermined period of time. Data also includes weight parameter X, to which a priority score is assigned (in this particular example a priority score of 90) if a patient's weight exceeds a predetermined value, symptom parameter Y to which a priority score is assigned (in this particular example a priority score of 90) if a patient reports symptoms, and co-morbidity parameter Z such as diabetes or the like to which a priority score is assigned (in this particular example a priority score of 20) if a patient has a co-morbidity such as diabetes. Accordingly, data for Patient 1 indicates that the patient has not been hospitalized within the predetermined time period, the patient's weight does exceed a predetermined weight, the patient has reported symptoms and the patient has diabetes. Data for Patient 2 indicates that the patient has been hospitalized within the predetermined time period, the patient's weight does not exceed a predetermined weight, the patient did not report symptoms and the patient has diabetes.

As noted above, some or all data points may be transmitted from the respective patient and received at the monitoring site and/or may be known data. For example, weight data and symptom data may be transmitted to the remote monitoring site from each patient and the other or additional diagnostic parameters may be stored in the patient's records, e.g., stored in a database, in communication with the remote monitoring site. Fr example, information related to hospitalizations and co-morbidities may be data that is stored in a database of patient's record. Regardless, the priority scores for each patient are summed to provide a patient status score for each patient. In this example, a status score of 200 is determined for Patient 1 and a status score of 95 is determined for Patient 2. The patient status scores are indicative of the status or severity of the patient's well-being or health and/or the risk for poor or worsening health, i.e., the patient's condition, and/or otherwise indicates the level or degree of urgency or lack thereof for evaluation. In this particular example, scoring is such that the patient with the higher patient status score (Patient 1) is in poorer health or has a greater risk for poor health or worsening condition or otherwise has a greater urgency for evaluation, than the patient with the lower patient status score (Patient 2). However, other scoring protocols may be used, e.g., protocols in which the lower scores are the highest priority, etc.

Once a patient status score is determined for each patient, the patients may be prioritized according to their status scores 6. In this manner, patients are prioritized according to the health status or severity of the patient's well-being or health relative to the other patients or risk for poor health or worsening condition relative to other patients. Once prioritized, patient data may be evaluated by one or more nurses at the remote site in the prioritized order. In other words, an evaluation queue that includes a plurality of patients may be produced, the order of each patient in the queue being based on the patients' status scores. The order of priority of the patients may be continuously or periodically updated, e.g., using a computer system, to add more patients and/or patient data for patients already prioritized as additional data is received at the remote monitoring site. In this manner, the priority of the patients for evaluation may be dynamic.

The priority order of patients may be in any suitable form, e.g., graphical form, list form, etc. In certain embodiments, a presentation list may be produced which includes the patients in the prioritized order. The list may be in any suitable form, e.g., may be printed on a substrate, such as paper or the like, etc. In certain embodiments, patient data may be queued in a computer system in the prioritized order that is accessible by a nurse for evaluation. The computer system may transmit or “feed” a nurse, e.g., to a nurse's computer or patient data viewing station, patient data in the prioritized order, e.g., one at a time. As described in greater detail below, a computer system may be used to process patient data and provide a patient status score for each patient. The computer system may be configured to store the data in a database. The computer may be configured to present the patients to a nurse's computer in a prioritized order so that the nurse may evaluate patient data. Evaluation by a nurse may result in an action such as a communication with the patient and/or patient's physician, notation in the patient's record, etc., or no action.

In certain embodiments, patient status scores may be determined using a plurality of data sets. An algorithm may be applied to the data sets to provide a patient status score. Such embodiments are described primarily with two data sets for exemplary purposes only and are in no way intended to limit the scope of the invention as it will be apparent that more than two data sets may be employed, e.g., three, four, five or more data sets may be employed. For example, a first status score may be determined using a first data set and a second data score may be determined using a second data set. An algorithm may be applied to the first and second status scores to provide a patient status score for use in prioritizing the patient in an evaluation queue.

The data points of each data set may be the same, different or some may be the same and some may differ. In certain embodiments, the data sets include different data. For example, a first data set may include data related to patient type parameters, diagnostic event parameters, medications, and medical conditions and a second data set may include measurement parameter data and symptom parameter data, e.g., information related to a patient's weight (such as in the monitoring of CHF patients) a patient's symptoms.

Embodiments of the subject invention include prioritizing CHF patients for evaluation by a nurse at a remote monitoring site using, e.g., two data sets. A first data set for CHF patients may include one or more of the following: (1) patient type parameters (e.g., whether the patient is a telephonically monitored patient, whether the patient is enrolled in a clinical study; (2) diagnostic event parameters (e.g., whether the patient has been hospitalized within a predetermined period of time such as within about the last 30 days and/or within about the last 120 days, whether review of the patient's information required a nurse to contact the patient and/or patient's physician, within a predetermined period of time such as within about the past 7 days, whether the patient has been temporarily absent for a clinical subset (e.g., too weak to stand, temporary illness, bereavement, and the like), whether the patient is newly enrolled in the monitoring program such as newly enrolled within about 1 to about 30 days and/or newly enrolled within about 31-120 days, whether the patient has returned from a temporary absence from the monitoring program such as within a predetermined period of time from return to the program such as within about 7 days from temporary absence, whether the patient is non-compliant such as whether the patient has not reported weight information for about 3 days or more, whether there has not been any communication with patient within a predetermined period of time such as no communication within about the last 21 days, and the like); (3) medications (e.g., whether the patient has started beta blockers within a predetermined time period such as within about the past 7 days, whether the patient has increased beta blocker dosage within a predetermined time period such as within about the past 7 days, whether the patient has begun a new, non-beta blocker pharmaceutical, and the like); (4) medical conditions or co-morbidities (e.g., whether the patient has one or more of diabetes, renal disease, COPD, asthma, chronic obstructive pulmonary disease (COPD), coronary artery disease, depression, and the like). Other diagnostic parameters may be used in addition to, or instead of, one or more o those described above. This data may be data recorded in a patient's record.

A second data set may include one or more of the following: weight parameters and symptom parameters. For example, weight parameters may include, e.g., whether a patient's weight exceeds a predetermined weight, the magnitude of weight change, and whether weight data has been reported, i.e., missing weight (i.e., no weight has been reported within a predetermined time period, e.g., for a daily monitoring cycle, no weight reported for two consecutive days). Symptom parameters may include, e.g., coughing, sleeplessness, water retention, and the like. Other diagnostic parameters may also be included in the first data set, e.g., whether a patient has been reviewed by a nurse at the remote monitoring site within a predetermined time period, e.g., within about the last three days. This data may be data received data transmitted from a patient.

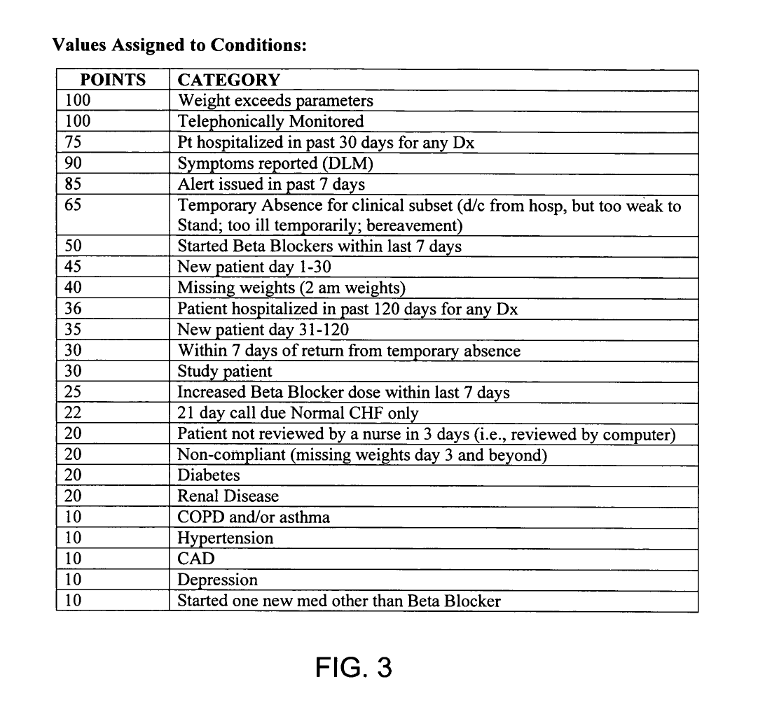

A priority score may then be assigned to each parameter for each data set. Exemplary priority scores for the above CHF parameters are shown in FIG. 3. As described above, once priority scores are assigned to each diagnostic parameter, an algorithm may be applied to the priority scores to provide a patient status score. For example, priority scores may be determined for the first set of diagnostic parameters and an algorithm may be applied to the priority scores to provide a first status score and priority scores may be determined for the second set of diagnostic parameters and an algorithm may be applied to the priority scores to provide a second status score. An algorithm may be applied to the first and second status scores to provide a patient status score. For example, an algorithm may include summing the first and second status scores to provide a patient status score. By way of example, using the priority scoring of FIG. 3, a first status score of 30 will be determined for a patient with a first data set that does not indicate recent hospitalization, but which indicates diabetes and hypertension as co-morbidities, and a second status score of 0 will be determined for the patient if their weight is not out of a predetermined range and no symptoms are reported. The patient status score or final score will be determined to be 30 (30+0). The score of 30 is then used to prioritize the review of the patient relative to other patients of the monitoring population.

The first and second status scores may be determined at the same or different times, where in certain embodiments they are determined at different times or at least independently of each other, e.g., as described below. The times at which an algorithm is applied to a data set to provide a status score may depend on a variety of factors, such as when and/or how the data is obtained at the remote monitoring site, the monitoring cycle (whether the patients are monitored daily, etc.), and the like.

As described above, in certain embodiments data for a data set (e.g., the first data set described above) may be may be obtained from a database of patient records, e.g., data may have been entered into a patient's record at the remote monitoring site by a nurse or staff thereof, which may be continually or periodically updated, e.g., during each monitoring cycle such as for example daily. Data for at least one other data set may be transmitted from each patient for receipt at a remote monitoring site, such as the second data set described above that includes relevant measurements such as weight measurements and/or symptom information. Accordingly, embodiments may include prioritizing patient data for review by a nurse at a remote monitoring site using data sets as follows. Each evening, e.g., from about 9 PM to about midnight, e.g., from about 10 PM to about midnight, each patient's records may be assessed and a status score may be provided to the parameters of each patient record. This may be accomplished manually or automatically by way of a computer system that includes a suitably programmed processor. The records may include diagnostic information described above as the first data set (e.g., may include patient type parameters, event parameters, medications, and co-morbidities). As some of the information of a patient's records may require updating since the previous assessment (e.g., the previous day), prior to determining a status score, the information may be updated which may be done manually by manual entry by a nurse or staff thereof and/or automatically by a computer system. In any event, a status score for this data set may be determined, e.g., each evening, and may be referred to as the nightly report.

The next consecutive day, the patients begin transmitting diagnostic data to the remote monitoring site. The data transmitted from each patient may include the information described above as the second data set (e.g., may include measurement parameters and symptoms), i.e., data that may have changed since the last prior nurse evaluation, e.g., data that may have changed over about the last 24 hours. For example, transmitted data may include weight information (raw or processed) and symptom information.

For example, in embodiments in which weight is a diagnostic parameter, e.g., for monitoring CHF patients, a patient may engage and activate a sensor such as a scale, e.g., step onto a scale, for obtaining diagnostic data about the patient such as measurement data. The sensor may be coupled to a processor, e.g., at the patient's site programmed to perform one or more of the following: capture data from the sensor; present a pre-selected series of questions to the patient and capture the patient's answers; and transmit the data and patient answers to a remote monitoring system and/or monitoring staff, as described in greater detail below. The processor may also be capable of comparing the data captured from the sensor with a preset target value, e.g., may be capable of comparing the captured data with preset minimum and maximum values; receiving instructional data from the remote monitoring system and effecting changes in the stored target value, minimum and maximum values, and question series presented to the patient in accordance therewith. The processor may be further capable of presenting the captured data and any variation from the target value to the patient; detecting and verifying proper operation of the system (self diagnostics); and accepting and verifying a patient's id code or passcode (if employed), and employing the questions, target value, and minimum and maximum values appropriate to the identified patient. In certain embodiments, the sensor and/or processor do not include a component which would allow a patient to self-analyze the collected data. In any event, the diagnostic data such as and weight data and/or symptom data and/or other information may be automatically transmitted from each monitored patient to a remote monitoring site as described below.

Accordingly, in certain embodiments that employ a daily monitoring cycle, the day following the nightly report the patients begin weighing themselves and transmit the data to the remote monitoring site. Data may be transmitted at any suitable time, where in certain embodiments the data is transmitted before about mid-day (e.g., before about 3 PM). A status score for this data set is determined and may be referred to as the interday report.

An algorithm is applied to these two status scores, the nightly report and the interday report, to provide a patient status score, e.g., the two scores may be summed. In cases in which a patient has not transmitted data prior to a predetermined time, e.g., prior to about mid-day, their nightly report status score may be used as the patient status score. The patients may be prioritized in an order based on their patient status score and presented to a nurse at the monitoring site, e.g., via a computer monitoring system thereof, in a manner so that the nurse is alerted to, or is otherwise made aware of, the priority order. For example, as described below, patient data may be forwarded to a nurse's computer monitor in the prioritized order, e.g., one patient or group of patients at a time or the like.

It will be apparent that patients may transmit data to the monitoring site at different times. As such, the priority order may be updated throughout the course of the day. For example, as patients weigh themselves during the time period for such and the weight information and any other information is transmitted to the remote monitoring site and a patient status score is determined, the patient's data may be integrated into the priority order so that a prioritized order of patients is maintained.

As described above, patient data may be presented to one or more nurses for evaluation in any suitable manner. In certain embodiments, an electronic or other list indicating the priority order of the patients may be compiled and presented to a nurse. In certain embodiments, patient data may be presented to a nurse using a computer-based system. In such embodiments, patient data is presented to a nurse's computer, e.g., from computer memory in communication with the nurse's computer, in the prioritized order. For example, a processor may be configured to transfer patient data stored in computer memory to a nurse's computer for evaluation in the prioritized order. Patient data may be continuously transferred to one or more computers (depending on the number of evaluation nurses) for evaluation in the prioritized order, until all of the patients in need of evaluation have been evaluated. For example, at the start of a monitoring shift, a nurse may turn on a computer and the patient of highest priority as determined above (e.g., having the highest patient status score) may be transferred from computer memory to the nurse's computer for evaluation. Once the nurse has evaluated the first patient's data and the nurse is available to evaluate another patient's data, patient data from the next highest priority patient in the prioritized queue (e.g., having the next highest patient status score) may then be transferred from computer memory to the nurse's computer for evaluation. This may be repeated until all of the patients' data has been evaluated.

This may be adapted for situations in which a plurality of nurses evaluate patient data. For example, the number of nurses available for evaluation of patient data may range from 1 to about 200 or more. In this regard, two nurses, for example, may be available for evaluating patient data. Each nurse may have a computer in communication with computer memory that includes the patient data. In such embodiments, patient data from the first patient in the prioritized order (e.g., having the highest patient status score) may be transferred to the first nurse for evaluation and patient data from the second patient in the prioritized order (e.g., having the next highest patient status score) may be transferred from to the second nurse for evaluation. Data from the next successive patient in the prioritized order may be transferred to the first or second nurse, depending on who is available for the next evaluation. This process may be repeated until all of the patients in the prioritized order have been evaluated by the nurses.

The above described methods of obtaining data at a remote site from a plurality of patients (e.g., CHF patients), assigning a priority score to the data received from each patient and applying an algorithm to the data using the priority score to obtain a patient status score, and presenting the patient status score to a healthcare professional for evaluation may be repeated a plurality of times, e.g., over a plurality of consecutive days. The number of consecutive days over which the methods may be repeated may vary depending on the particular monitoring protocol, where the methods may be repeated over about 30 or more consecutive days, e.g., over about 60 or more consecutive days, e.g., over about 365 consecutive days or more, in certain embodiments. Data obtained over the consecutive days may be plotted on a graph or the like for each of the plurality of patients for evaluation by a healthcare professional.

As described above, the subject invention may be employed in the remote monitoring of a diverse number of different patient conditions. For example, the subject invention may be used in the monitoring of drug titration where a physiological parameter can be measured and related to the effect of a drug that is being self-administered, e.g. insulin, anticoagulants, ACE inhibitors, beta-blockers, and the like. Of interest is the use of the subject invention in the remote monitoring of patients having cardiac associated diseases, such as CHF.

Systems

The subject invention also includes systems for performing the subject methods. Embodiments includes systems for prioritizing patient data from a plurality of patients and include a remote monitoring system having a processor or plurality of processors configured to receive and process the patient data to provide a patient status score for each patient based on the data, a plurality of patient data input modules in communication with the processor, and communication means configured to transmit data from the patient data input modules to the processor. The term “processor” refers to any hardware and/or software combination which will perform the functions required of it. For example, any processor may be a programmable digital microprocessor such as available in the form of a mainframe, server, or personal computer (desktop or portable). Where the processor is programmable, suitable programming may be communicated from a remote location to the processor, or previously saved in a computer program product (such as a portable or fixed computer readable storage medium, whether magnetic, optical or solid state device based). For example, a magnetic or optical disk may carry the programming, and may be read by a suitable disk reader communicating with each processor (if more than one). Systems adaptable for use with the subject invention include, e.g., those described in U.S. Pat. No. 6,080,106, the disclosure of which is herein incorporated by reference.

Patient Data Input Module

The patient data input module or patient interface component of the system may include both a sensor and an active interrogation means, i.e. a “question and answer” means. As described above, a system may include a plurality of patient input modules such that data from each patient may be transferred via communication means to the remote monitoring site.

The sensor is capable of measuring a parameter diagnostic of the patient's health. As used herein, a “sensor” is any device capable of measuring a physical characteristic or attribute of the patient, e.g., a parameter diagnostic of the patient's health or condition. For example, the sensor may be any device that is capable of measuring such parameters, such as a scale for measuring a patient's weight, or a device for measuring the patient's blood pressure, oxygen saturation, ECG, blood glucose level, and the like. For systems designed for use in the monitoring of cardiac associated diseases, particularly CHF, the sensor may be either: (1) a device for sensing edema, or (2) a weight detection device, such as a scale.

A data input module may also include an active interrogation component or “question and answer” component for presenting the patient with one or more questions related to the patient's health status and recording the patient's answers to the questions. Questions which may be presented by the interrogation means may be simple “yes” or “no” questions, or be more complicated, requiring a more descriptive response or a selection of one of a plurality responses. The particular questions presented to the patient by the interface system will be chosen in view of the particular condition being monitored, or the state or progression of the condition being monitored and may include question described above.

The interrogation component may take any convenient form. For example, it may be an input panel, where the input panel may be a toggle switch or pair of push buttons (e.g., “Yes/No”), or may include a full keyboard or touch-screen input system. Alternatively, the interrogation component may include a microphone, which may be coupled with voice-recognition software. The interrogation component may be configured to provide information and/or questions to the patient, e.g., visually and/or audibly. The questions may be displayed on one or more of a standard CRT, LED or LCD display, or played audibly over a speaker.

In certain embodiments, the patient data input module will be automatic, by which is meant that the patient does not need to take any active steps to activate the module, e.g. the patient does not have to turn the module “on.” For example, where the sensor is a scale, the patient need only step onto the scale. The input module will then sense the presence of the patient on the scale, determine and record the patient's weight, and automatically present to the patient one or more questions and record the patient's answers thereto.

The patient data input module may further include a processor. The processor may be any processor having sufficient power to store and present data, and may range from a microprocessor to a personal computer. The processor may processes the data collected by the patient, e.g., collected by the sensor of the system.

In use, the system may be activated by standing on the scale, by physically turning on the system, or by entering a passcode on the patient data input means, e.g., the input panel. A series of questions may be asked of the patient. For example, the patient may be asked if he or she ate a meal prior to being weighed. If the patient answers affirmatively, the patient may then be questioned about the size of the meal. If the patient answers negatively, he or she may be questioned about the length of time elapsed since the last meal (or the approximate time of the last meal). The patient may be asked if the correct medication was taken in a timely manner: if so, the processor proceeds on to the next topic; if not, the processor may begin a series of questions to determine why the medication was skipped.

Communication Means

The system also includes communication means, which may transmit data to, and/or receive data from, a remote monitoring system. As such, the communication means may include both an output means for transferring the collected and processed data to a remote monitoring system and an input means for receiving instructional data from a remote monitoring system. The communication means may be any device or system capable of transmitting the data (measured parameters and patient answers) to the remote monitoring system, physician or staff, and receiving data (new target values, questions) from the remote monitoring system, e.g. for storage. Suitable communication means include, but are not limited to, computer readable storage medium, a modem, serial interface, LAN, WAN, wireless transmitter, MICROWIRE, I²C, +5 V serial, 1-Wire® interface, General Purpose Interface Bus (“GPIB”), RS-232, Peripheral Component Interconnect (“PCI”), memory mapped I/O and Universal Serial Bus (“USB”), and the like.

The input means will generally include a serial or parallel port to the processing means, but may alternatively or additionally include a keyboard or numeric pad, disc drive, and the like. The input means may be used by the healthcare professional at the remote monitoring site to effect changes in the system's programming, for example altering a target weight value, changing the question series, or selecting an alternate question series or language.

Remote Monitoring System

Embodiments of the remote monitoring system of the subject invention may be generally characterized as a computer-based system which includes a processor configured to receive patient data from a plurality of patient input modules and process and prioritize the patient data, e.g., to provide patient status scores. The processor may also be configured to present the patient data to a nurse evaluation station in the prioritized order, e.g., one at a time, for example from memory in which the patient data is stored. The remote monitoring system may also include a database for storing patient data, e.g., a database of patient records.

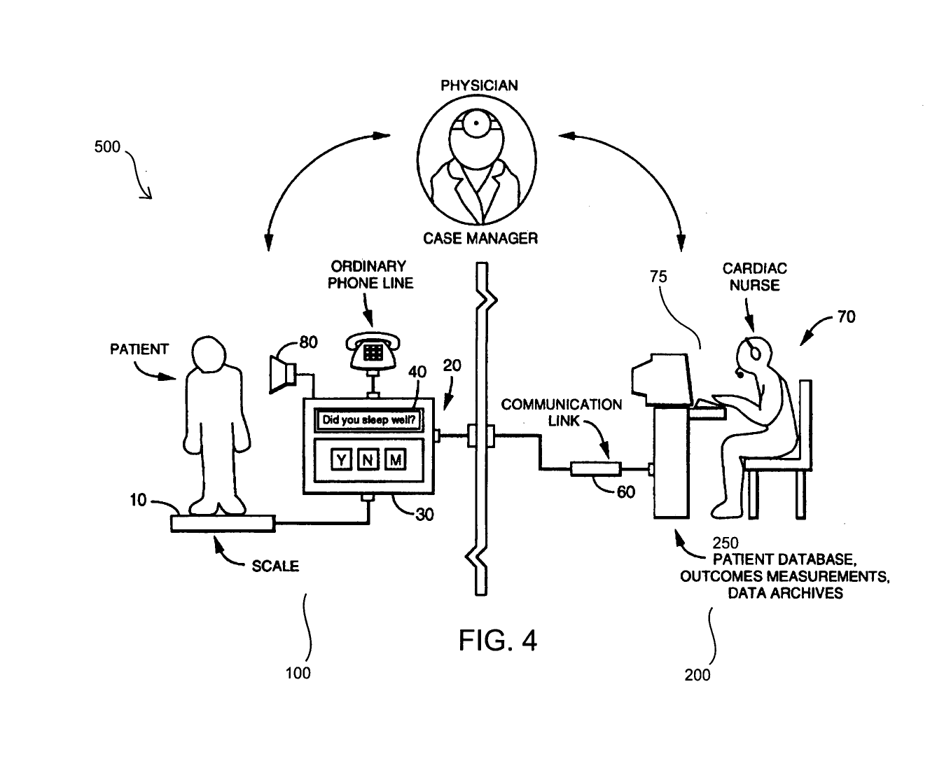

FIG. 4 shows a schematic of an exemplary embodiment of a subject system 500. The system includes patient input module 100 in communication with a remote monitoring site system 200. The remote monitoring site system includes computer system 250 for storing and processing patient data and which is in communication with one or more evaluation stations 75. As described above, a system may include a plurality of patient input modules, wherein each may be in communication with the remote monitoring site system.

A sensor 10, herein shown as a scale, provides a digital output to processor 20, which calculates the difference between the patient's weight and the preset target weight, and outputs the difference on display 40. If the processor detects that the weight indicated by the scale is below a preset minimum weight (or above a preset maximum weight), the processor does not initiate the question sequence, and may optionally displays a warning (e.g., “Unauthorized Use”) on display 40. Processor 20 then presents a series of predetermined questions to the patient through display 40 and/or speaker 80. The patient then responds by keying in answers on keyboard 30, while processor 20 records the answers. At the completion of the question series, processor 20 transmits the recorded answers through communications link 60 to a computer system 250 at the remote monitoring site.

The computer system at the monitoring site is configured to apply an algorithm to the patient data to prioritize the data, e.g., to assign priority scores to the data of each patient, sum the priority scores and provide a patient status score for each patient. The computer system may then transfer patient data to each evaluation station 75 in a prioritized order.

Computer Readable Media

One or more aspects of the subject invention may be in the form of computer readable media having programming stored thereon for implementing the subject methods. The computer readable media may be, for example, in the form of a computer disk or CD, a floppy disc, a magnetic “hard card”, a server, or any other computer readable media capable of containing data or the like, stored electronically, magnetically, optically or by other means. More specifically, computer readable medium may include stored programming embodying an algorithm for carrying out the subject methods. Accordingly, such a stored algorithm is configured to, or is otherwise capable of, determining patient status scores for a plurality of patients, as described above. Stored programming embodying steps for carrying-out the subject methods may be transferred to a computer such as a personal computer (PC), (i.e., accessible by a researcher or the like), by physical transfer of a CD, floppy disk, or like medium, or may be transferred using a computer network, server, or other interface connection, e.g., the Internet.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1.-9. (canceled)

10. The method of claim 30, wherein said determining further comprising for each patient:
(a) weighting said transmitted data and summing said weighted received data to provide a first status score;
(b) weighting said additional data and summing said weighted additional data to provide a second status score; and
(c) summing said first and second status scores to provide said patient status score.

11. The method of claim 30, repeating steps (a)-(c) a plurality of times over a plurality of consecutive days.

12. The method of claim 11, wherein the plurality of consecutive days is thirty or more days.

13. The method of claim 30, wherein said determining is performed by a processor.

14. The method of claim 30, further comprising storing said patient data in a data storage unit.

15. The method of claim 14, wherein said data storage unit is computer memory.

16. The method of claim 30, wherein each of said plurality of patients has a condition.

17. The method of claim 16, wherein said condition is a cardiac condition.

18. The method of claim 17, wherein said cardiac condition is congestive heart failure.

19.-29. (canceled)

30. A method for prioritizing patient data from a plurality of patients for review by a healthcare professional, said method comprising:
(a) receiving data at a remote monitoring site, said data transmitted from a plurality of patients;
(b) determining a patient status score for each patient at different times throughout the course of each day based on said transmitted data, wherein each patient is assigned a numerical status score over a range of from 1 to 500 by assigning priority scores to each diagnostic parameter and summing said scores wherein said priority scores are weighted and review of the patient's scores is prioritized relative to other patients; and
(c) presenting said data to a healthcare professional for evaluation in an order based on said determined scores;
wherein said received data comprise a plurality of diagnostic parameters comprising weight data, symptom data and additional data comprising information on whether the patient has been hospitalized within a predetermined period of time, medication data and co-morbidity data, wherein said additional data is stored in a database.
